(19) United States
(12) Patent Application Publication
(10) Pub. No.: US 2023/0225610 A1
(43) Pub. Date: Jul. 20, 2023

(54) APPARATUS FOR TESTING AGE-RELATED MACULAR DEGENERATION AND METHOD OF USING THE SAME

(71) Applicant: THE REGENTS OF THE UNIVERSITY OF MICHIGAN, Ann Arbor, MI (US)

(72) Inventors: Jacob Lutz, Ann Arbor, MI (US); Vichal Palandira Muthanna, Ann Arbor, MI (US); Andrew Yu, Ann Arbor, MI (US); Mario Russo, Cambridge, MA (US); Rachel Sun, Ann Arbor, MI (US); Yannis Paulus, Ann Arbor, MI (US)

(21) Appl. No.: 18/008,429

(22) PCT Filed: Jun. 4, 2021
(86) PCT No.: PCT/US21/35927

§ 371 Date: Dec. 5, 2022

Related U.S. Application Data
(60) Provisional application No. 63/037,669, filed on Jun. 11, 2020.

(51) Int. Cl. A61B 3/032; A61B 3/00; G16H 10/60

(52) U.S. Cl. CPC A61B 3/032; A61B 3/005; G16H 10/60

(57) ABSTRACT

A portable ocular screening device for tracking macular degeneration having a portable body including a graduated focusing mechanism including an inner chamber. The apparatus also includes a monocular eyepiece disposed on the graduated focusing mechanism and a display screen and a processor disposed within the inner chamber. Additionally, a method of monitoring macular degeneration includes providing a monocular screening device having a display screen disposed within a body of the screening device. The method further includes displaying an image and also providing a prompt in response to the display of the image. Subsequently, the method includes receiving a response via an input device, the response indicative of a user's perception of the image and evaluating the response to determine if the response indicates a user accurately perceived the first image.

### FIELD OF THE INVENTION

This application relates generally to apparatus and techniques for testing for degenerative eye disease, and more particularly, to apparatus for testing age-related macular degeneration and methods of using the same.

### BACKGROUND

Age-related macular degeneration is a degenerative disease of the eye affecting the retina. Additionally, age-related macular degeneration is the leading cause of adult blindness in industrialized countries and currently affects approximately 190 million people worldwide. When left untreated, age-related macular degeneration causes central vision loss. However, there is a narrow window of time when treatment can be administered to prevent the progression of age-related macular degeneration to adult blindness.

Age-related macular degeneration is classified in two distinct stages: The first stage is dry age-related macular degeneration. The second stage is wet age related macular degeneration. The transition from dry age-related macular degeneration to wet age-related macular degeneration can happen spontaneously. Wet age-related macular degeneration is responsible for more than 80% of severe vision loss when not treated within one to two weeks following the transition from dry to wet age-related macular degeneration.

Various techniques exist for evaluating the progression of age-related macular degeneration. The current standard of care for treating age-related macular degeneration involves periodic physician assessments. However frequent visitations are untenable due to the high costs and burdens of extensive physician visits. Yet, due to the spontaneous nature of the transition from dry to wet age-related macular degeneration, infrequent evaluations could result in extensive vision loss.

### BRIEF DESCRIPTION OF THE DRAWINGS

FIG. 1 is a perspective view of a portable ocular screening device of a first embodiment of the present disclosure for testing age-related macular degeneration and measuring a transition between dry and wet age-related macular degeneration;

FIG. 2 is an exploded view of the example portable ocular screening device of FIG. 1;

FIG. 3 is a perspective view of a second embodiment of a portable ocular screening device of the present disclosure for testing age-related macular degeneration, having a single actuatable interface component;

FIG. 4 is a perspective view of third embodiment of a portable ocular screening device of the present disclosure for testing age-related macular degeneration, having a toggle-button interface component;

FIG. 5a is a plan view of a display including a graphical user interface of the portable ocular screening device for initiating a screening test;

FIG. 5b is a plan view of a display including a graphical user interface displaying a test image for user evaluation;

FIG. 5c is a plan view of a display including a graphical user interface displaying a request for a response from the user based on the user's observation of the test image;

FIG. 6a is a plan view of a test image graphic of a distorted circle; and

FIG. 6b is a plan view of a test image graphic of a perfect circle.

### DETAILED DESCRIPTION

The current standard of care, that requires patients to regularly meet with physicians for observation of age-related macular degeneration, is burdensome and insufficient given the spontaneity of the ocular degeneration. The gap in the medical care requires an affordable and effective remote monitoring solution to track the progression from dry to wet age-related macular degeneration. Such a remote monitoring system would increase patient agency in monitoring their own retinal health while also decreasing the burden on the healthcare system and making doctor visits more efficient and profitable.

To overcome the burden on the healthcare system and provide improved patient agency, the present invention involves a portable ocular monitoring device for monitoring age-related macular degeneration. This portable ocular monitoring device is a handheld monocular screening device usable by patients in their own homes at regular intervals (e.g., daily, semiweekly, or weekly). Preferably, the patient will use of the portable ocular monitoring device on a daily basis. As a result, the device is able to track the progression from dry to wet age-related macular degeneration.

The retina test of the present invention relies on the concept of shape discrimination hyperacuity (SDH). SDH is a measure of a person's ability to detect sinusoidal deformations from circularity. Wet age-related macular degeneration affects the ability to identify distortions in common shapes. Patients who have wet age-related macular degeneration can be identified based on their minimum distortion-detection threshold (MDDT). In order to test a patient's MDDT, the patient is shown a series of circle-like images, some perfectly circular and some distorted to be non-circular. After each image is displayed the user is asked whether the image was a perfect circle. The accuracy of the responses is a measure of the user's MDDT.

The patient's MDDT data is collected and stored over a sequence of trials and statistically analyzed to determine a change in a patient condition. For example, a patient with lowering MDDT scores may be indicative of a transition from dry to wet age-related macular degeneration. As the portable ocular monitoring device relies on stored data over a series of trials, the more regular and consisted the trials, the more accurate the results. In some examples, the data on the patient's MDDT scores can influence future circle-like images to provide distorted, non-circular images that will best evaluate a patients MDDT. By adjusting the distortion of the circle-like images, the portable ocular monitoring device can converge on a user's MDDT during a single test, and longitudinally over several weeks and months.

Additionally, the portable ocular monitoring device is designed as a monocular monitoring device. The device is designed to test only one eye at a time so that each eye can be tested individually, and if the eyes are degenerating asymmetrically the better eye does not compensate for the degenerating eye.

The present invention of the portable ocular monitoring device is capable of (1) remotely and accurately identifying the transition of dry age-related macular degeneration to wet age-related macular degeneration; (2) being readily incorporated into the daily routine of the user/patient; and (3) effectively reducing the burden, on the patient and the healthcare system, of expensive and repetitive appointments with physicians, including retina specialists.

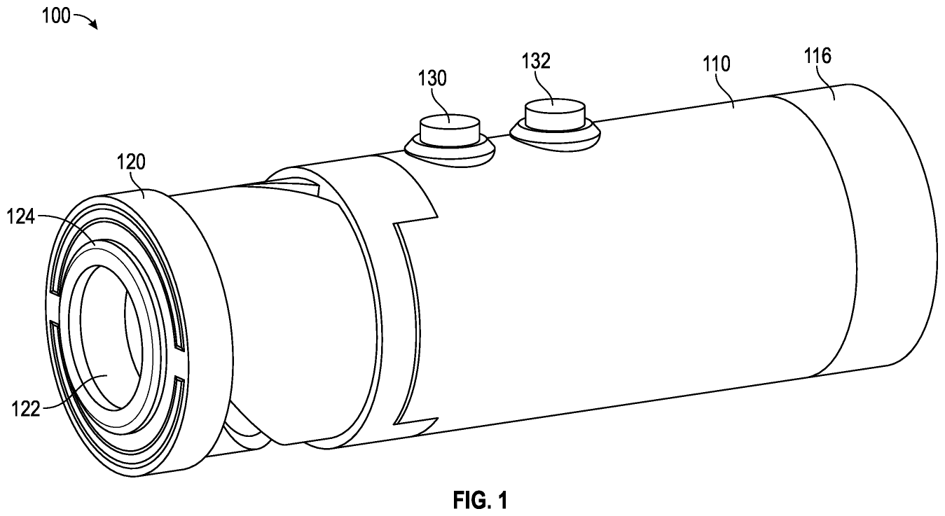

FIG. 1 is a perspective view of a first embodiment of a portable ocular screening device 100 of the present disclosure for testing age-related macular degeneration and measuring a transition between dry and wet age-related macular degeneration. The portable ocular screening device 100 includes a portable body 110. Additionally, connected on a first end of the portable body 110 is a graduated focusing mechanism 112 having an inner chamber 114 (not shown). Opposite the first end of the portable body 110, the portable body 110 is connected to a cap 116. The portable body 110 of the portable ocular screening device 100 is designed to be lightweight and sized to be handheld.

Connected to the graduated focusing mechanism 112, the portable ocular screening device 100 includes a monocular eyepiece 120. The monocular eyepiece 120 allows a patient to look into the inner chamber 114 with only one eye. Disposed in the inner chamber is a display screen (discussed in greater detail below). As discussed above, the monocular eyepiece 120 is designed to accommodate only one eye so the patient can only test one eye at a time. As a result, the monocular eyepiece prevents a second eye from compensating for any degeneration in the first eye and skewing the test results for the first eye. Testing each eye separately also permits identification of asymmetric macular degeneration.

In some embodiments within the scope of the present disclosure, the monocular eyepiece 120 includes a magnification lens 122 surrounded by foam padding 124 for patient comfort. In some embodiments, the magnification lens 122 is a 22 millimeter (mm) biconcave lens that generates a 10× (10 times) magnification. In other embodiments, the magnification may be greater or lesser, depending on the patient and/or the display used in the inner chamber 114. Additionally, the graduated focusing mechanism 112 can be adjusted to focus the magnification lens 122 on the display disposed in the inner chamber 114. For example, as illustrated in FIG. 1 the graduated focusing mechanism 112 includes helical grooves that both allow the graduated focusing mechanism 112 to be secured to the portable body 110 and twisted to adjust the distance between the magnification lens 122 and the display. In other embodiments, the graduated focusing mechanism can be adjusted and secured by any known means. The adjustment of the graduated focusing mechanism allows patients with different visual acuities to focus on the screen based on their personal needs.

Additionally disposed on the portable body 110 of the example portable ocular screening device 100 are manually actuatable interface components. As further illustrated in FIG. 1, the manual actuatable interface components include a first button 130 and a second button 132. The first button 130 and the second button 132 are illustrated as protruding from the portable body 110. However, in other examples, the first button 130 and the second button 132 may be flush with the portable body 110, or even recessed into the portable body 110. The patient, when looking through the monocular eyepiece 120 can control the portable ocular screening device 100 by using the first button 130 and the second button 132 in response to prompts on the display disposed in the inner chamber 114. In other embodiments, the manually actuatable interface components may be more or fewer buttons, switches, toggles, knobs, directional pads (D-pad), thumbsticks, or other suitable interfaces.

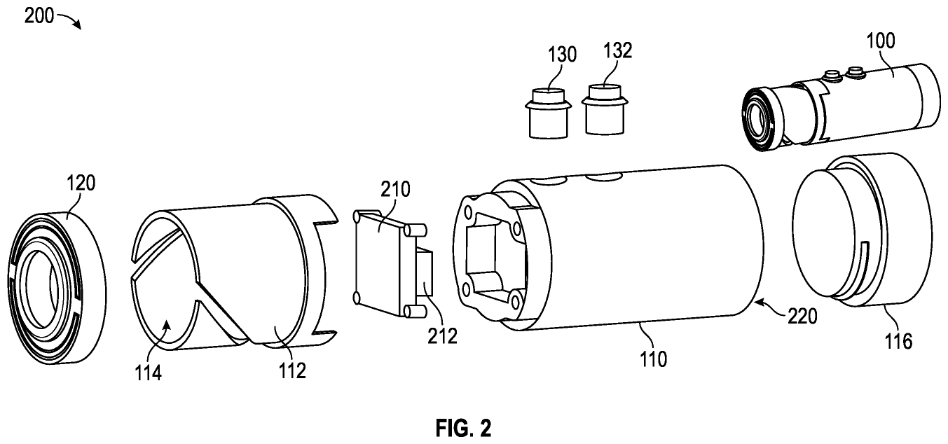

FIG. 2 is an exploded view 200 of the embodiment of the portable ocular screening device 100 of FIG. 1. When assembled, the components of the exploded view 200 form the portable ocular screening device 100, as illustrated in FIG. 1. As illustrated in FIG. 2, the portable ocular screening device 100 includes a display 210 disposed in the inner chamber 114. The display 210 is typically a 1.5 inch display. In other examples, the display can be larger or small than 1.5 inches. The display can be, for example, an organic light-emitting diode (OLED). In other examples, the display could be a quantum light-emitting diode (QLED), Micro light-emitting diode (MicroLED), liquid crystal display (LCD), or other displays able to fit within the inner chamber 114.

Additionally included in the inner chamber 114 is the processor 212. The processor may be a single-board computer (e.g., a Raspberry Pi 3) or may be a more complex system. In the example of the portable ocular screening device 100, the processor 212 controls the display 210 and receives inputs from the manual actuatable interface components (e.g., first button 130 and second button 132). Additionally, the processor 212 is able to process the responses from the patient to measure a patients MDDT. After the processor 212 processes responses, the processor stores responses and MDDT scores in a memory (not shown) disposed in the portable ocular screening device.

The example portable ocular screening device 100 additionally includes an additional cavity 220 in the portable body 110. The cavity 220 is covered by cap 116 which can be removed from the portable body 110 to provide access to the cavity 220. The cavity 220 can include a battery power source (not shown) for powering the display 210 and the processor 212. The cavity can additionally include access to the processor 212 and interfaces (not shown) for connection to external computer systems, external power sources for charging batteries, or other interface components.

FIG. 3 is a perspective view of a second embodiment of a portable ocular screening device 300 for testing age-related macular degeneration having a single actuatable interface component. In the embodiment illustrated in FIG. 3, the portable ocular screening device 300 also includes a portable body 310 and a monocular eyepiece 320. In contrast to the portable ocular screening device 100 of FIGS. 1 and 2, the portable ocular screening device 300 includes a single manual actuatable interface component instead of the example first button 130 and second button 132. The single button 330 is the only manual actuatable interface component of the portable ocular screening device 300. A single button 330 may be beneficial to avoid confusion on the part of the user as to which actuatable interface component to manipulate in response to a prompt seen on a graphical user interface of a display of the portable ocular screening device 300 during use.

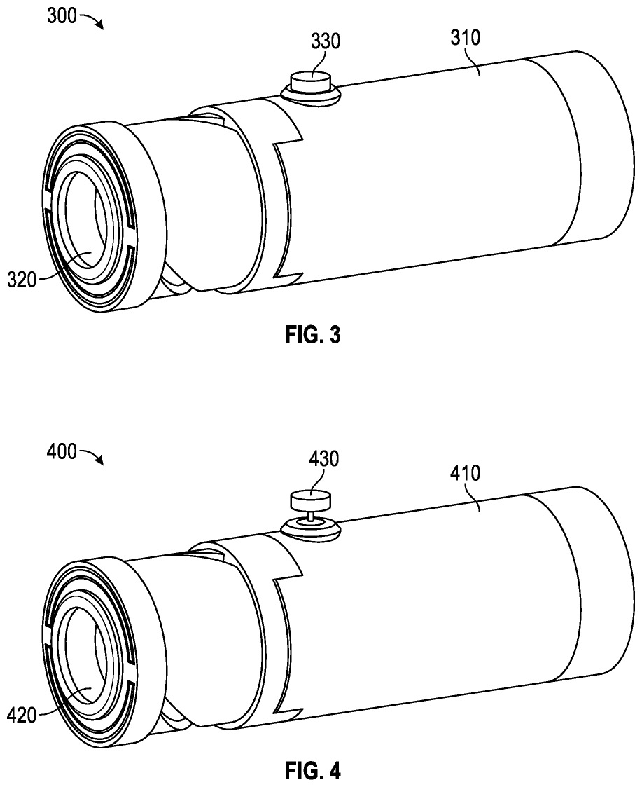

FIG. 4 is a perspective view of a third embodiment of a portable ocular screening device 400 for testing age-related macular degeneration, having a toggle-button interface component. The portable ocular screening device 400 also includes a portable body 410 with a monocular eyepiece 420. However, the portable ocular screening device 400 includes a toggle-button 430. This toggle-button 430 serves as the manual actuatable interface component, and can, in some examples, be both pivoted to move a cursor on a display and depressed similar to a button. In some examples the pivoting of the toggle-button is limited to movement in only a first and second direction, or alternatively, the toggle-button may be one that can be moved in any direction.

The manual actuatable interface components of the embodiments of the portable ocular screening devices illustrated in FIG. 1 (i.e., first button 130 and second button 132), FIG. 3 (i.e., single button 330), and FIG. 4 (i.e., toggle-button 430) are examples of manual actuatable interface components. Any comparable button or toggle is considered as useable with the portable ocular screening device of the present invention. Additionally, any combination of buttons or toggles is considered within the scope of with the present invention.

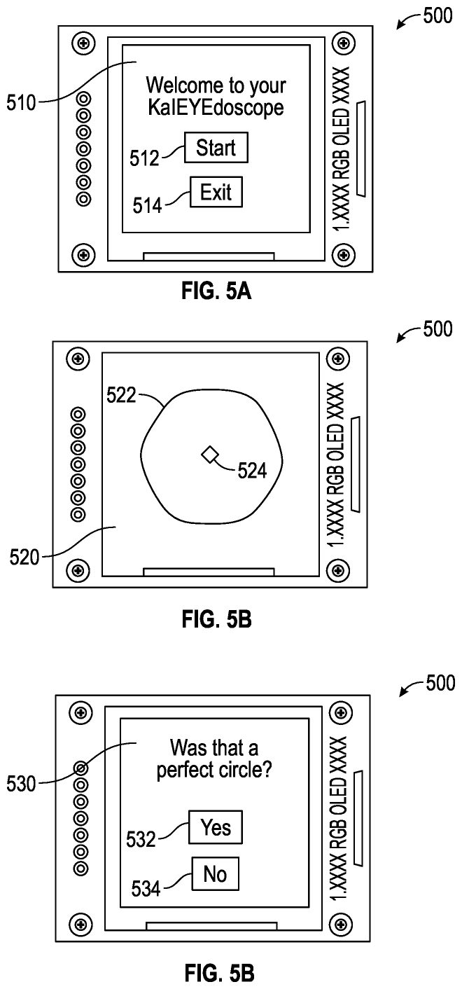

FIG. 5a is a plan view of a display 500 presenting an example graphical user interface 510 of the portable ocular screening device 100 for initiating a screening test. By way of the graphical user interface 510, a patient is prompted to choose one of two options. The first option 512 is to initiate an exam (as indicated by the selectable “Start” prompt). By way of example only, the first option 512 may be shaded with a first color associated with the first button 130. As a result, the patient can associate the selection of the first option 512 with the operation of the first button 130 based on the similarity of the color. Additionally, the graphical user interface 510 includes a second option 514. In some examples, the second option is shaded with a second color associated with the second button 132.

In alternative examples, the patient can utilize a toggle. In such an example, the first option 512 can be selected by moving a toggle in a first direction. Accordingly, the second option can be selected by moving the toggle in a second direction. Additional user interface options are also considered including using a single button and preset timed periods for selecting options via the single button.

FIG. 5b is a plan view of the display 500 presenting an example graphical user interface 520 including a test image 522 for user evaluation. In the example of FIG. 5b, the test image 522 is an image of a distorted circle with a fixation point 524 located centrally on the display 500. In other examples, the test image 522 may be an image of a perfect circle instead of a distorted circle. The test image is displayed for a preset period of time. The test image 522 is displayed for 0.5 seconds. In other examples, the test image 522 may be displayed for more time (e.g., approximately 2 seconds) or less time (e.g., approximately 0.1 second).

FIG. 5c is the display 500 presenting an example graphical user interface 530 requesting a response from the patient based on the test image 522. In the example graphical user interface 530, the request is, “Was that a perfect circle?” Accordingly, the patient may select a first response 532 of “Yes,” or a second response 534 of “No.” In some examples, the first response 532 may be shaded with a first color associated with the first button 130 and the second response 534 may be shaded with a second color associated with the second button 132. However, the response may be selected differently based on the manual actuatable interface components on the portable ocular screening device.

The selected response is processed by the processor 212 and stored in memory. In the example of FIGS. 5b and 5c, a patient selecting the second response 534 would improve their MDDT score while a patient selecting the first response 532 would decrease their MDDT score, because the test image 522 was not a perfect circle. After selection of a response, the process repeats and another test image is displayed to the patient for the same preset period of time as the test image 522. If a user accurately identifies a distorted, non-circular image, the processor may provide a less distorted, non-circular image to evaluate the patients minimum distortion-detection threshold (MDDT). Alternatively, if a patient incorrectly identifies a distorted, non-circular image, the processor may provide a more distorted, non-circular image. By adjusting the distortion of the circular image, the processor can converge upon the patient's MDDT and accurately calculate a shape discrimination hyperacuity (SDH) value for the patient for the test.

Each of the selected responses is processed by the processor 212 and stored in memory. The processor 212 is also able to, from the collection of responses, calculate a trend based on a plurality of the patient's MDDT and SDH scores, and thus, measure progression of dry to wet age-related macular degeneration.

After the processor has collected sufficient information on a first eye (e.g., has converged on an SDH score), the processor will indicate, by way of the graphical user interface and/or by an audible signal, and/or by a light or other indicator on the surface of the portable ocular screening device, that the user should begin a test of a second eye. The testing of the second eye will progress in the same manner as the first eye, but only the second eye will be able to see into the portable ocular screening device 100.

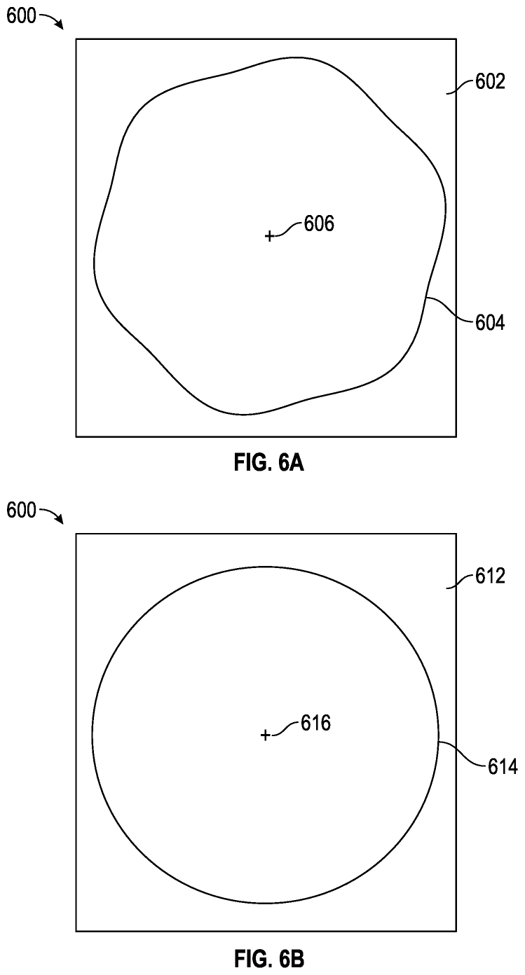

FIG. 6a illustrates an example test image graphic 602 of a distorted circle 604 on a graphical user interface 600. In other examples, the distorted circle could be more distorted or less distorted than shown in FIG. 6a. In the example of FIG. 6a, the example test image graphic 602 includes a fixation point 606 located centrally in the graphical user interface 600. The fixation point 606 is a pair of perpendicular lines, however the fixation point 606 could be a dot, small circle, or other shape centered in the graphical user interface 600.

FIG. 6b illustrates an example test image graphic 612 of a perfect circle 614 on a graphical user interface 610. In the example of FIG. 6b, the example test image graphic 612 includes a fixation point 616 located centrally in the graphical user interface 610. The fixation point 616 is a pair of perpendicular lines, however the fixation point 616 could be a dot, small circle, or other shape centered in the graphical user interface 610. Additionally, the fixation point 616 could be different from the fixation point 606 of FIG. 6a.

As illustrated in FIGS. 6a and 6b, the graphical user interfaces 600 and 610 are shown with a black background and the fixation points 606 and 616, the distorted circle 604, and the perfect circle 614 are shown in white. This contrast reduces eye strain, however other color schemes and contrasts are also considered within the scope of the invention.

While various embodiments have been described herein, modifications can be made thereto that are still considered within the scope of the appended claims.

What is claimed is:

1. A portable ocular screening device comprising:
a portable body including a graduated focusing mechanism including an inner chamber;
a monocular eyepiece disposed on the graduated focusing mechanism;
a display screen disposed within the inner chamber; and
a processor positioned within the inner chamber.

2. The portable ocular screening device of claim 1, further including a power source disposed in the portable body.

3. The portable ocular screening device of claim 1, further including a manually actuatable interface component.

4. The portable ocular screening device of claim 3, wherein the manually actuatable interface component is a mechanical interface selected from the group consisting of a depressible button, a pair of depressible buttons, a toggle-button, a switch, a directional pad, and a knob.

5. The portable ocular screening device of claim 3, wherein the processor is electronically connected to the display screen and the power source and responsive to actuation of the manually actuatable interface component.

6. The portable ocular screening device of claim 1, wherein the monocular eyepiece includes a magnification lens.

7. The portable ocular screening device of claim 6, wherein the graduated focusing mechanism adjusts a distance between the magnification lens and the display screen.

8. A method of monitoring vision acuity related to macular degeneration, comprising:
providing a portable monocular screening device having a display screen disposed within a body of the monocular screening device;
displaying, via the display screen, a first image;
providing, via the display screen, a first prompt in response to the display of the first image;
receiving a first response via an input device on the portable monocular screening device, the response indicative of a user's perception of the first image; and
evaluating the first response to determine if the first response indicates a user accurately perceived the first image.

9. The method of monitoring vision acuity of claim 8, further including:
displaying, via the display screen, a second image, different from the first image;
providing, via the display screen, a second prompt in response to the display of the second image;
receiving a first response via the input device on the portable monocular screening device, the response indicative of the user's perception of the second image; and
evaluating the first response and the second response to determine if the first response and the second response are consistent with responses indicative of the user having macular degeneration.

10. The method of monitoring vision acuity of claim 9, further including generating a metric measuring a shape discrimination hyperacuity value based on evaluating at least the first response and the second response.

11. The method of monitoring vision acuity of claim 9, wherein in displaying the first and second images, the first image is one of an image of a circle or a distorted image of a circle and the second image is the other of the image of the circle or the distorted image.

12. The method of monitoring vision acuity of claim 9, further including the portable monocular screening device providing additional images and collecting additional responses until a shape-discrimination hyperacuity threshold is established.

13. The method of monitoring vision acuity of claim 12, further including prompting a user to test a second eye.

14. The method of monitoring vision acuity of claim 8, further including storing the first response in a memory located in the portable monocular screening device.

15. The method of monitoring vision acuity of claim 8, further including storing the first response in an external memory.
